(19) United States
(12) Patent Application Publication
(10) Pub. No.: US 2003/0212357 A1
(43) Pub. Date: Nov. 13, 2003

(54) Method and apparatus for treating wounds with oxygen and reduced pressure

(76) Inventor: Edgar Alan Pace, Pinnacle, NC (US)

Correspondence Address: Charies Y. Lackey, Post Office Box 5871, Winston-Salem, NC, 27113-5871, US

(21) Appl. No.: 10/142,324

(22) Filed: May 10, 2002

(51) Int. Cl. A61F 13/00; A61F 15/00

(52) U.S. Cl. 602/41

(57) ABSTRACT

A method of and apparatus for treating wounds by applying oxygen and reduced pressure and/or other medication sufficient in time and magnitude to induce healing and closure of the wound. The method and apparatus are applicable to wounds, infected wounds, and live tissue attachments. Wound treatment apparatus includes an impermeable wound cover sealably positioned over the wound. A screen of material such as open-cell foam or rigid porous material is placed beneath the wound cover and over the wound. A vacuum pump supplies suction within the wound cover and over the treatment site to provide oxygen, other medication and reduced pressure directly to the wound.

### 1. FIELD OF THE INVENTION

The present invention relates to apparatus and method for treating wounds by applying oxygen, medication and reduced pressure directly to the wound.

### 2. BACKGROUND OF THE INVENTION

The treatment of open wounds that are too large to spontaneously close has long been a troublesome area of medical practice. Closure of an open wound requires inward migration of surrounding epithelial and subcutaneous tissue. Some wounds, however, are sufficiently large or infected that they are unable to heal spontaneously. In such instances, a zone of stasis in which localized edema restricts the flow of blood to the epithelial and subcutaneous tissue forms near the surface of the wound. With insufficient blood flow, the wound is unable to successfully fight bacterial infection and is accordingly unable to close spontaneously.

An initial stage of wound healing is characterized by the formation of granulation tissue which is a matrix of collagen, fibronectin, and hyaluronic acid carrying macrophages, fibroblasts, and neovasculature that forms the basis for subsequent epithelialization of the wound. Infection and poor vascularization hinder the formation of granulation tissue within wounded tissue, thereby inhibiting wound healing. It therefore becomes desirable to provide a technique for increasing blood circulation within wounded tissue to promote spontaneous healing and to reduce infection.

Poor blood circulation and infection at the wound may also hinder attachment of skin grafts or flaps upon wounded tissue. Skin grafts and flaps will not attach to tissue that is poorly vascularized, infected or necrotic. However, grafts and flaps can be used with much greater success on tissue that, although wounded, is able to form granulation tissue. Accordingly, a technique for promoting blood circulation at the wounded tissue would also promote successful attachment, or “take,” of skin grafts or flaps to the wounded tissue as a consequence of increased blood circulation within the grafts or flaps.

Another problem encountered during the treatment of wounds is the selection of an appropriate technique for wound closure during the healing process. Sutures are often used to apply force to adjacent viable tissue in order to induce the edges of a wound to migrate together and heal. However, sutures apply a closure force to only a very small percentage of the area surrounding a wound. When there is scarring, edema, or insufficient tissue, the tension produced by the sutures can become great causing excessive pressure to be exerted by the sutures upon the tissue adjacent to each suture. As a result, the adjacent tissue often becomes ischemic thereby rendering suturing of large wounds counterproductive. If the quantity or size of the sutures is increased to reduce the tension required of any single suture, the quantity of foreign material within the wound is concomitantly increased and the wound is more apt to become infected. Additionally, the size or type of a particular wound may prevent the use of sutures to promote wound closure. It therefore becomes desirable to provide an apparatus and method for closing a large wound that distributes a closure force evenly about the periphery of the wound.

Wounds resulting from ischemia, or lack of blood flow, are also often difficult to heal since decreased blood flow to a wound may inhibit normal immune reaction to fight infection. Patients that are bedridden or otherwise non-ambulatory are susceptible to such ischemic wounds which become decubitus ulcers or pressure sores. Decubitus ulcers form as a result of constant compression of the skin surface and underlying tissue thus restricting circulation. Since the patient is often unable to feel the wound or to move sufficiently to relieve the pressure, such wounds can become self-perpetuating. Although it is common to treat such wounds with flaps, the conditions that initially caused the wound may also work against successful flap attachment. Wheelchair-bound paraplegics, for example, must still remain seated after treatment of pelvic pressure sores. It therefore becomes desirable to provide a treatment procedure for ischemic wounds that can be conducted in situ upon an immobile or partially mobile patient.

Other types of wounds in which ischemia leads to progressive deterioration include partial thickness burns. A partial thickness burn is a burn in which the cell death due to thermal trauma does not extend below the deepest epidermal structures such as hair follicles, sweat glands, or sebaceous glands. The progression of partial thickness burns to deeper burns is a major problem in burn therapy. The ability to control or diminish the depth of burns greatly enhances the prognosis for burn patients and decreased morbidity resulting from burns. Partial thickness burns are formed of a zone of coagulation, which encompasses tissue killed by thermal injury, and a zone of stasis. The zone of stasis is a layer of tissue immediately beneath the zone of coagulation. Cells within the zone of stasis are viable, but the blood flow is static because of collapse of vascular structure due to localized edema. Unless blood flow is re-established within the zone of stasis soon after injury, the tissue within the zone of stasis also dies. The death of tissue within the zone of stasis is caused by lack of oxygen and nutrients, reperfusion injury (re-establishment of blood flow after prolonged ischemia), and decreased migration of white blood cells to the zone resulting in bacterial proliferation. Again, it becomes desirable to provide a technique for treating burn wounds by enhancing blood circulation to the wounded tissue to inhibit burn penetration.

The treatment of wounds, ulcers, burns and skin grafts by reduced pressure at the wound is fully disclosed in U.S. Pat. No. 5,636,643, the entire contents of which are incorporated herein by reference. While this technique has been very successful in treating these problems, it is desired to carry such treatment to even higher levels. It is to that goal that the present invention is directed.

### SUMMARY AND OBJECTIVES OF THE INVENTION

It has been shown that treating wounds by applying reduced pressure; i.e., pressure that is below ambient atmospheric pressure, can be very effective in inducing wounds to heal and close. The technique has been applied to open wounds, infected wounds, ulcers, burns and skin grafts. The present invention significantly enhances that technology by improving the quality and reducing the time period of the healing process and includes subjecting oxygen and/or other medication directly against the wound while simultaneously applying reduced pressure at the wound site. This multiple application of oxygen, medication and reduced pressure provides exceptional rapid healing, increased formation of granulation tissue, closure of chronic open wounds, reduction of bacteria density within wounds inhibition of burn penetration and enhancement of flap and graft attachment.

The apparatus of the present invention comprises a reduced pressure application device which is applied to a wound and to normal tissue surrounding the wound. The device includes a fluid impermeable wound cover for covering and enclosing the wound and sealing means for sealing the wound cover to the surrounding tissue of the wound to maintain reduced pressure in the vicinity of the wound during wound treatment. When the cover is sealed in position over the wound, a generally fluid-tight or gas-tight sealed enclosure is formed over the wound site. The sealing means may be an adhesive applied to the underside of the wound cover or may be a separate sealing member such as an adhesive strip or sealing ring for positioning the cover edge around the periphery of the wound. The device also includes a suction port for supplying reduced pressure within the sealed volume enclosed beneath the wound cover. The suction port may be a nipple on the wound cover or a tube feedthrough beneath the wound cover. The device may also include a porous wound screen for placement in the wound or in a position overlaying the wound in order to prevent overgrowth of wound tissue during treatment. The porous wound screen may be a sponge or open cell foam material for placement in the wound.

Oxygen and/or other medication is introduced to the wound through a port or ports in the wound cover or through a tube feedthrough beneath the wound cover or through an another exhaust port if reduced pressure is not being applied. Oxygen and other substance flow into the wound exhausts through the suction port which applies the reduced pressure under the wound cover. Oxygen or other medication sprays or mists may be injected into a reduced pressure atmosphere by controlling or metering the injection rate such that it remains less than the displacement capacity of the vacuum pump at the desired reduced pressure. A control valve may be installed in the oxygen or other medication supply lines going into or under the wound cover. Oxygen and other medication flow are metered into the reduced pressure enclosure and compared to the evacuation flow rate out the suction port to maintain the desired reduced pressure within the reduced pressure enclosure. When wound treatment requires alternate application and non-application of reduced pressure and atmospheric pressure (off and on or pulsating action), oxygen and other medications may still be injected under the sealed cover by an automatic valve in the cover or suction tube to allow these substances to flow into and through the cavity under the cover without producing enough positive pressure to break the cover seal over the wound. This feature allows a septic and healthy atmosphere to surround the wound even when the reduced pressure is not being applied to the wound.

From the foregoing, it is apparent that a principal objective of the present invention is to provide a method for treating wounds which comprises applying a negative or reduced pressure along with a metered amount of oxygen and/or other medication flow over the wound and an area sufficient to promote the migration of epithelial and subcutaneous tissue toward the wound for a time period sufficient to facilitate closure of the wound.

Another objective of the present invention is to provide a method of treating a burn wound which comprises applying a negative or reduced pressure along with a metered amount of oxygen and/or other medication to the burn over an area and for a time sufficient to inhibit progression in the depth of the burn. The method is useful on a partial thickness burn soon after its infliction.

Yet another objective of the present invention is to provide a method of treating tissue damage which comprises applying a negative or reduced pressure and a metered amount of oxygen and/or other medication to a wound for a time sufficient to reduce bacterial density in the wound. One use of this method is its application to a wound for a selected time period such as at least three days to reduce the bacterial density of an infected wound to the point at which surgical closure can be attempted.

Still another objective of the invention is to provide a method of enhancing the attachment of adjacent tissue to a wound which comprises applying negative or reduced pressure, oxygen and perhaps other medications to a joined complex of the adjacent living tissue and the wound at a sufficient magnitude of reduced pressure and for a sufficient time duration to promote the migration of epithelial and subcutaneous tissue toward the complex. This method enhances attachment of adjacent tissue to tissues of the wound edges. This method also enhances the attachment of an open skin graft to the wound tissue.

Yet a further objective of the present invention is to provide apparatus for carrying out the enumerated methods for treating wounds, infections, burns and grafts.

Thus there has been outlined the more important features of the invention in order that the detailed description that follows may be better understood and in order that the present contribution to the art may be better appreciated. There are, of course, additional features of the invention that will be described hereinafter and which will form the subject matter of the claims appended hereto. In that respect, before explaining at least one embodiment of the invention in detail, it is to be understood that the invention is not limited in its arrangement of the components set forth in the following description and illustrated in the drawings. The invention is capable of other embodiments and of being practiced and carried out in various ways.

It is also to be understood that the phraseology and terminology employed herein are for the purpose of description and should not be regarded as limiting in any respect. Those skilled in the art will appreciate that the concept upon which this disclosure is based may readily be utilized as a basis for designing other structures, methods and systems for carrying out the several purposes of this development. It is important that the claims be regarded as including such equivalent methods and products resulting therefrom that do not depart from the spirit and scope of the present invention. The application is neither intended to define the invention, which is measured by its claims, nor to limit its scope in any way.

Thus, the objects of the invention set forth above, along with the various features of novelty which characterize the invention, are noted with particularity in the claims annexed to and forming a part of this disclosure. For a better understanding of the invention, its operating advantages and the specific results obtained by its use, reference should be made to the following detailed specification taken in conjunction with the accompanying drawings wherein like characters of reference designate like parts throughout the several views.

The drawings are included to provide a further understanding of the invention and are incorporated in and constitute a part of this specification. They illustrate embodiments of the invention and, together with their description, serve to explain the principles of the invention.

### BRIEF DESCRIPTION OF THE DRAWINGS

FIG. 1 is a schematic, elevational and sectional view of a wound treatment apparatus in accordance with the present invention in which a reduced pressure device which includes a fluid impermeable wound cover is sealed over the wound, oxygen is introduced into the sealed area near the wound, and a vacuum system provides reduced pressure within the wound cover and induces oxygen to flow out from the sealed area without breaking the wound cover seal;

FIG. 2 is a schematic sectional elevational view of a reduced pressure appliance in accordance with another embodiment of the present invention having a rigid, fluid impermeable wound cover seal over the wound and a rigid or semi-rigid screen overlying the wound with oxygen and other medication being injected into the covered wound area;

FIG. 3 is a schematic sectional elevational view of another embodiment of the present invention having a semi-rigid fluid impermeable cover enclosing the wound and a rigid or semi-rigid screen overlying the wound with an overlying flexible film impermeable cover sheet sealing the enclosure over the wound, and oxygen and other medication are being metered into the covered area; and

FIG. 4 is a schematic sectional elevational view of another embodiment of the present invention having a rigid outer frame with support legs for supporting a flexible, fluid impermeable sealing cover over the wound with oxygen being metered into the wound covered area.

### DETAILED DESCRIPTION OF THE PREFERRED EMBODIMENTS

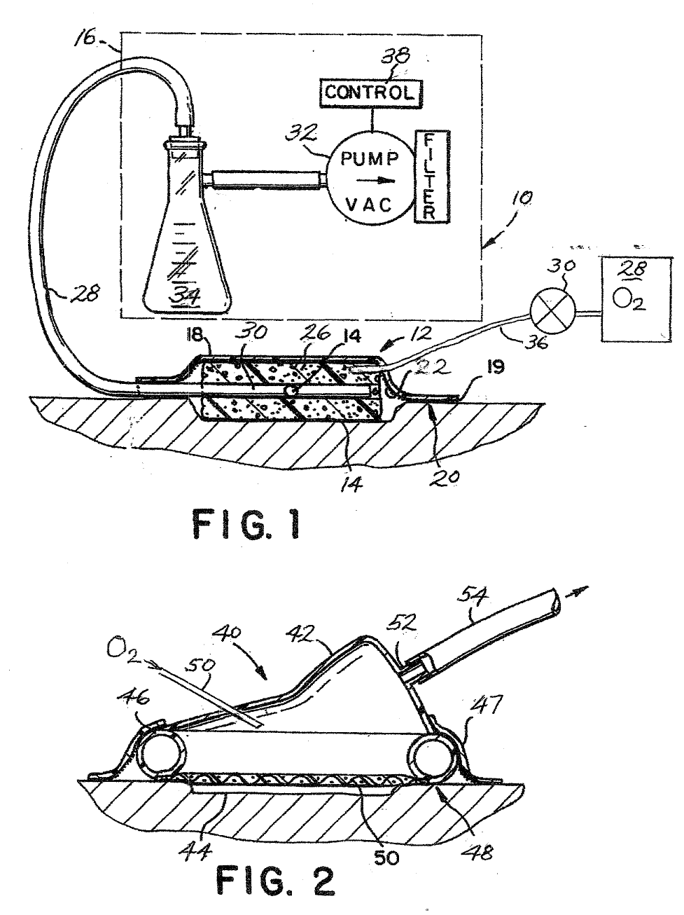

Referring now to the drawings and particularly to FIG. 1, a wound treatment apparatus generally designated 10 is shown having a reduced pressure device 12 for enclosing a wound site to provide a fluid- tight or gas-tight enclosure over the wound site to effect treatment of a wound 14 with reduced or negative pressure. The reduced pressure device 12 is applied to and sealed over the wound site in order to enclose the wound site for treatment with suction or reduced pressure within a sealed generally fluid-tight or gas-tight enclosure. For creating suction within device 12, it is connected with a vacuum system, generally designated 16, to provide a source of suction or reduced pressure for device 12 at the wound site. Device 12 includes a fluid-impermeable wound cover 18 in the form of a flexible, adhesive fluid permeable polymer sheet for covering and enclosing wound 14 and the surrounding skin at the wound site. The wound cover 18 includes adhesive backing 20 which functions to seal the wound cover to the normal skin 22 around the periphery of wound 24 to provide a generally gas-tight or fluid-tight enclosure over wound 14. Cover 18 must have sufficient adhesion to form a fluid-tight or gas-tight seal 24 around the periphery of the wound and to hold the cover 18 in sealed contact with the skin during the application of suction or reduced or negative pressure.

Device 12 also includes a porous wound screen 26 which is placed within wound 14. Wound screen 26 is placed over substantially the expanse of the wound to prevent its overgrowth. The size and configuration of wound screen 26 can be adjusted to fit the individual wound and can be formed from a variety of porous materials. It may be in the form of an open-cell polymer foam such as a polyurethane foam which is sufficiently porous to allow gas flow to or from wound 14. As shown in FIG. 1, screen 26 is cut to an appropriate shape and size to fit within wound 14.

Device 12 also includes a suction port in the form of a hollow suction tube 28 that connects with vacuum system 16 to provide suction within the sealed enclosure. Suction tube 28 serves as a suction port for device 12 and also functions to induce the flow of oxygen and/or other gaseous or atomized medication across wound 14 and away from the sealed area which will be discussed in greater detail subsequently. An end segment 30 of tube 28 is embedded within foam screen 26 for providing suction or reduced pressure within the enclosure formed under wound cover 18. The open cells of foam screen 26 facilitate gas and/or mist flow through the enclosure. In addition, foam screen 26 functions to prevent wound overgrowth and to hold wound cover 18 generally out of contact with wound 14 during the application of suction within the enclosure.

Wound treatment is further enhanced in the present embodiment by the provision of an oxygen supply 28 which is carefully controlled and metered by an automatic valve 30. Flexible tube 36 is extended through the wound cover 12 and into foam screen 26 so that oxygen can be metered therein with or without the presence of reduced pressure in the enclosure. Additional healing properties are also contemplated since other medications such as antibiotics, pain suppressors, hormones and dietary supplements are capable of being atomized and applied with or without oxygen on and around the wound within the reduced pressure area.

Vacuum system 16 includes a suction pump 32 that produces a source of reduced pressure or suction which is supplied to reduced pressure device 12 by suction tube 28. As shown in FIG. 1, a fluid trap generally designated 34, is interconnected between suction pump 32 and device 12 to remove and collect any exudate which may be aspirated from wound 14 by reduced pressure device 12. Device 12 functions to actively draw fluid or exudate from the wound. Collection of exudates in fluid trap 34 intermediate pump 32 and device 12 is desirable to prevent clogging of the pump.

Predetermined amounts of suction or reduced pressure are produced by vacuum pump 32 which is preferably controlled by a control device 38 such as a switch or timer which may be set to provide cyclic on/off operation of vacuum pump 32 according to user-selected intervals. Alternatively, vacuum pump 32 may be operated continuously without the use of a timer. Vacuum system 16 has other safety features such as a system shutdown should exudate aspirated from wound 14 exceed a predetermined quantity. To protect the site of wound 14 from impact or abrasion during treatment, a reduced pressure device employing a rigid or semi-rigid wound cover may be utilized over the site of the wound. As shown in FIG. 2, a reduced pressure device 40 includes a CPR mask 42 that provides a rigid wound cover for enclosing an appropriately sized wound 44. Mask 42 is impermeable to fluids or gases so that a fluid-tight or gas-tight enclosure is effectively formed over the wound site. Mask 42 is sufficiently rigid to support itself away from wound 44 during the application of suction or reduced pressure so that it does not collapse into wound 44. CPR mask 42 is of the type having an inflatable air cuff 46 around the base of the mask. Cuff 46 may be inflated by an external valve for sealing mask 42 against normal skin 48 around the periphery of wound 44. Air cuff 46 also prevents the base of the mask from digging into skin 48 during the application of reduced pressure. While the suction created within mask 42 may be sufficient to hold the device in position by causing air cuff 46 to seal to skin 48, more effective attachment to the surrounding skin 48 may be obtained by the use of a strip of fluid impermeable adhesive material 47 secured to skin 48 and to the base of cuff 46.

In FIG. 2, an oxygen input tube 50 extends through mask 42 in a sealable fashion so that oxygen may be discreetly injected into the sealed opening near wound 44. A suction port serving as a hose connector 52 has a suction tube 54 attached. The other end of tube 54 is connected with vacuum system 30. Here again wound treatment is through the application of reduced pressure within the sealed opening over the wound and the introduction of discreet amounts of oxygen. Connector 52 and tube 54 force the reduced pressure within the sealed area of the wound and induce oxygen flow through tube 50, across the wound and out of the sealed area. Oxygen flow exhaust may also be accomplished by using a separate port (not shown) extending through the wall of mask 42.

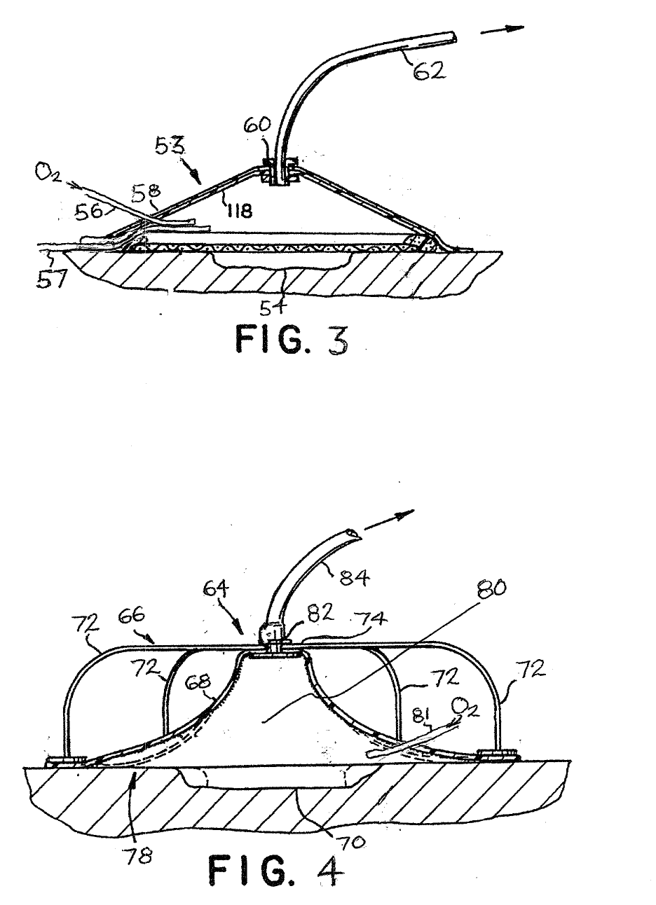

In FIG. 3, a reduced pressure appliance 53 is depicted for enclosing and treating a wound 54 with suction or reduced pressure, and oxygen is carefully metered into the sealed area next to the wound by tubing 56 extending through cover sheet 58 into the sealed area. Reduced pressure and oxygen removal is again handled by a connector 60 and tube 62 going to vacuum system 16. Other medication is also introduced into the sealed area through another tube 57 placed between the skin surrounding the wound and cover sheet 58.

Where a downward pressure into the wound is not desired, a reduced pressure device 64 like that shown in FIG. 4 may be utilized having a support structure 66 which is positioned external to a flexible sealing sheet 68 for covering wound 70. Device 64 shown in FIG. 4 includes a series of spider-like legs 72 radiating outwardly from a central support hub 74. Legs 72 hold central support hub 74 over wound 70. The flexible sealing sheet 68 is adhered to the connector 76 at hub 74 into the surrounding normal skin 78 so that the sheet is suspended over wound 70 from the hub 74 in a tent-like manner.

Oxygen can be introduced into the formed closure 80 by a sealed entry tube 81 through cover sheet 68 in the manner shown. In this embodiment, reduced pressure and removal of flowing oxygen is through connector 82 and tubing 84.

Utilizing a negative pressure appliance and oxygen directly against the wound can dramatically reduce the healing time normally required when only negative pressure is utilized. Supplying reduced pressure to the appliance in an intermittent or cyclic manner has also been demonstrated to be useful for treating wounds. The introduction of oxygen and/or other medication into the wound enclosure can be similarly operated synchronously with the activation and de-activation of reduced pressure or oxygen and/or other medication can be applied even when no reduced pressure is in place. A reduction in bacteria density in the wound by more than 50% is attainable.

The present invention also includes a method of treating damaged tissue which comprises the steps of applying negative pressure and oxygen and/or other medication to a wound for a selective time and at a selective magnitude sufficient to reduce bacterial density.

The invention also includes a method of treating a burn which comprises the steps of applying negative pressure and oxygen and/or other medication to the burn over an area with pre-determined reduced pressure and for a time sufficient to inhibit formation of a full thickness burn.

The present invention also provides a method for enhancing the attachment of living tissue to a wound which comprises the steps of first joining the living tissue to the wound to form a wound-tissue complex, then applying a negative or reduced pressure of selective magnitude and oxygen and/or other medication to the wound-tissue complex over an area sufficient to promote migration of epithelia and subcutaneous tissue toward the complex with the negative pressure being maintained for the selected time and oxygen and/or other medication being simultaneously employed to facilitate closure of the wound.

There may be many modifications, alterations and changes without departing from the scope or spirit of the essential characteristics of the invention. It is thus clearly understood that the above embodiments are only illustrative and not restrictive in any sense. The scope and period of the present invention are limited only by the -terms of the appended claims.

What is claimed is:

1. A method of treating a wound comprising the steps of: applying a reduced pressure to the wound which includes positioning an impermeable cover over the wound, the cover having an input port and a suction port; sealing the periphery of the cover to tissue surrounding the wound; introducing oxygen to the wound through the input port; connecting the suction port to a vacuum system for producing the reduced pressure; and maintaining the oxygen and reduced pressure to the wound until the wound begins to heal.

2. A method of pretreating a skin flap to promote attachment of the flap to a wound comprising the step of applying oxygen and reduced pressure to a region of skin tissue adjacent the wound prior to the detachment of the skin tissue adjacent the wound to form the flap from the region of skin.

3. The method as claimed in claim 1 further comprising: introducing medication to the wound and maintaining the medication to the wound until the wound begins to heal.

4. The method as claimed in claim 1 wherein the selected stage of healing is cessation of partial thickness burn progression.

5. The method as claimed in claim 1 wherein healing in the wound results in a reduction in bacteria density in the wound by more than 50%.

6. A method of promoting attachment of a skin graft onto a wound comprising the steps of: attaching the graft to the wound; and applying oxygen, medication and reduced pressure to the graft to promote blood circulation to the graft.

7. The method as claimed in claim 6 wherein the graft is a skin flap, the method further comprising the steps of: applying oxygen, medication and reduced pressure to a region of skin adjacent the wound; and forming the flap by detaching the skin from the region prior to the attaching step.

8. Apparatus for administering oxygen, medication and a reduced pressure to a wound comprising: an impermeable cover covering and enclosing the wound to maintain oxygen and reduced pressure at the site of the wound; a seal sealing the cover to tissue surrounding the wound; oxygen, medication and reduced pressure supply means for connection to a source of oxygen, medication and suction, the reduced pressure supply means cooperating with the cover to supply reduced pressure beneath the cover; and a screen adapted to prevent outgrowth of wound tissue, the screen being located between the wound and the cover.

9. The apparatus as claimed in claim 8 wherein the screen is a porous sheet.

10. The apparatus as claimed in claim 8 wherein the seal includes an adhesive material on the cover securing the cover to the tissue surrounding the wound.

11. The apparatus as claimed in claim 8 wherein the screen is a foam sheet.

12. The apparatus as claimed in claim 8 wherein the oxygen, medication and reduced pressure supply means includes a screen having an open cell foam and at least one segment of tubing embedded within the screen.

13. Apparatus for promoting closure of a wound comprising: an impermeable cover covering the wound; adhesive means on the cover forming a seal between the cover and tissue surrounding the wound; support means supporting the cover outward from the wound forming an enclosed volume bounded by the cover, the wound and tissue surrounding the wound; supply means supplying oxygen and reduced pressure to the enclosed volume deforming the cover to exert tension upon the tissue surrounding then wound.
